(19) United States
(12) Patent Application Publication
(10) Pub. No.: US 2005/0075609 A1
(43) Pub. Date: Apr. 7, 2005

(54) Protective needle clips

(76) Inventor: Patrick C. Latona, Wernersville, PA (US)

Correspondence Address: CHRISTIE, PARKER & HALE, LLP, PO BOX 7068, PASADENA, CA, 91109-7068, US

(21) Appl. No.: 10/677,810

(22) Filed: Oct. 1, 2003

(51) Int. Cl. A61M 5/178

(52) U.S. Cl. 604/164.08

(57) ABSTRACT

Protective needle clips are discussed having multiple engagement arms and biasing forces for blocking a needle tip of a needle and for securing onto the needle. Exemplary needle clips include those having an engagement arm having an opening that moves from a radially inwardly position to a radially outwardly position when the needle moves proximal of the opening. Additional arms may be incorporated with each arm having an opening for allowing the needle to project their through. As the needle moves proximal of the first opening of the first engagement arm, the openings of the remaining engagement arms wedge against a side of the needle to secure the needle clip to the needle.

Protective needle clips are generally discussed herein for shielding needle tips of hypodermic needles to prevent accidental contact therewith with particular discussion relating to protective needle clips comprising radially actuating spring clip portions for shielding the needle tips.

### BACKGROUND

Protective needle clips are well known in the art for use in conjunction with hypodermic needles. Broadly speaking, a typical prior art protective needle clip is mounted over a hypodermic needle. The prior art protective needle clip typically has a body and at least one resilient portion, which may be inherently resilient or is caused or urged to be resilient by an exterior resilient member, such as a spring.

After an injection, the hypodermic needle is withdrawn from a body and cause to travel relative to the needle clip so that the needle clip moves from a proximal position on the needle to a distal position on the needle where the needle tip is located. When the needle clip reaches the distal end near the needle tip, the at least one resilient portion is caused to move radially inwardly over the needle tip to block the needle tip. Other mechanisms are also used with the prior art needle clip to either activate the resilient portion, to launch the needle clip, to retain the needle clip on the needle, etc., which are not discussed for simplicity.

Although prior art needle clips provide workable options for health care workers, there is a continuing need for an improved and/or alternative needle clip. Accordingly, disclosed herein are improved alternative needle clips having multiple engaging arms and biasing forces to secure the same onto hypodermic needles to shield the needle tips from accidental contact therewith. Also disclosed are methods for using and making the same.

### SUMMARY

The present invention provides protective needle clips. More particularly, the present invention may be practiced by providing a protective needle clip comprising a first engaging arm, a connecting base extending at a first angle from the first engaging arm, a second engaging arm extending at a second angle from the connecting base, a connecting top extending at a third angle from the second engaging arm, and a third engaging arm extending at a fourth angle from the connecting top; wherein each of the first, second, and third engaging arms comprises an opening, and wherein at least a portion of the third engaging arm is disposed in between the first engaging arm and the second engaging arm.

Alternatively, the present invention may be practiced by providing a protective needle clip comprising an engaging arm comprising an opening extending from a connecting base, the engaging arm and the connecting base comprising a ready position, which is a position in which a needle comprising a needle tip passes through the opening of the engaging arm and biasing the connecting base and the at least a portion the engaging arm radially inwardly relative to the needle, and an activated position, which is the position in which the needle tip moves proximal of the opening and the connecting base and the at least a portion of the engaging arm move radially outwardly away from the needle.

Relatedly, a method for shielding a needle tip of a hypodermic needle is disclosed comprising moving the hypodermic needle relative to a protective needle clip such that the protective needle clip moves from a proximal position on the needle to a distal position on the needle, the protective needle clip comprising a first engaging arm extending from a connecting base, a second engaging arm extending from said connecting base, a connecting top extending from the second engaging arm, and a third engaging arm extending from the connecting top; wherein the first, second, and third engaging arms each comprises an opening and wherein the hypodermic needle extends through the three openings; moving the needle tip of the hypodermic needle proximal of the opening of the first engaging arm; and allowing at least a portion of the first engaging arm to move radially outwardly away from the needle.

### BRIEF DESCRIPTION OF THE DRAWINGS

These and other features, aspects and advantages of the present invention will be more fully understood when considered with respect to the following detailed description, appended claims and accompanying drawings, wherein:

FIG. 1 is a semi-schematic perspective view of an exemplary protective needle clip provided in accordance with aspects of the present invention;

FIG. 2 is a semi-schematic side view of the protective needle clip of FIG. 1;

FIG. 3 is a semi-schematic side of the protective needle clip of FIG. 1 in a ready position over a hypodermic needle;

FIG. 4 is a semi-schematic side view of the protective needle clip of FIG. 1 in an activated position over the hypodermic needle;

FIG. 5 is a semi-schematic side view of an alternative protective needle clip in an activated position over a hypodermic needle provided in accordance with aspects of the present invention,

FIG. 6 is a semi-schematic cross-sectional side view of a catheter needle assembly comprising a modified spring clip;

FIG. 7 is a semi-schematic end view of the catheter needle assembly of FIG. 6 taken along line F7-F7; and

FIG. 8 is a semi-schematic perspective view of the spring clip of FIG. 6.

### DETAILED DESCRIPTION

The detailed description set forth below in connection with the appended drawings is intended as a description of the presently preferred protective needle clip embodiments provided in accordance with aspects of the present invention and is not intended to represent the only forms in which the present invention may be constructed or utilized. The description sets forth the features and the steps for constructing and using the protective needle clips of the present invention in connection with the illustrated embodiments. It is to be understood, however, that the same or equivalent functions and structures may be accomplished by different embodiments that are also intended to be encompassed within the spirit and scope of the invention. Also, as denoted elsewhere herein, like element numbers are intended to indicate like or similar elements or features.

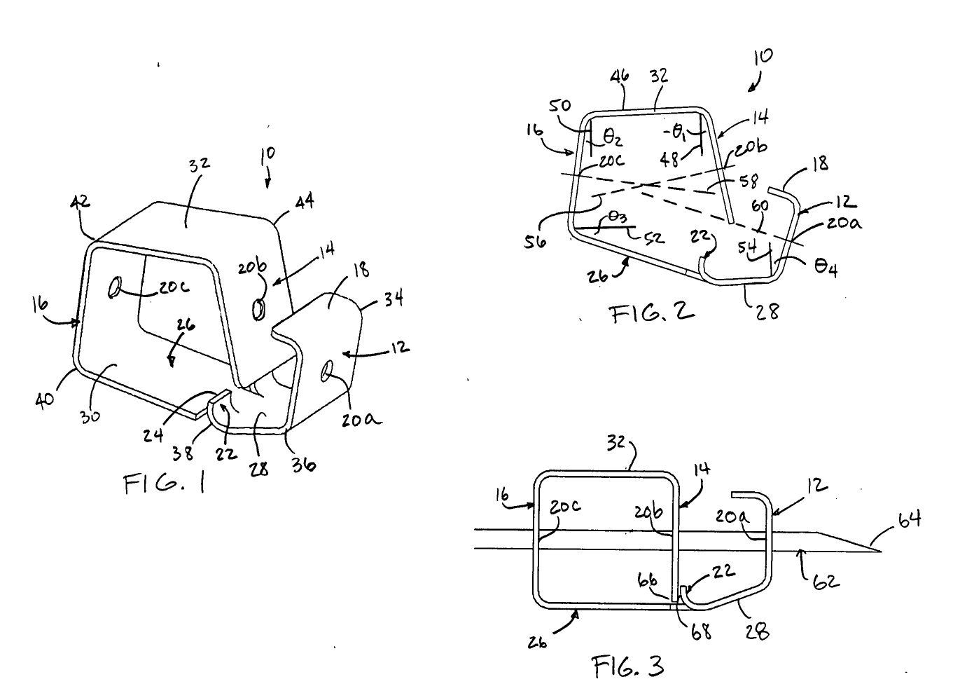

A semi-schematic perspective view of a protective needle clip provided in accordance with aspects of the present is shown in FIG. 1, which is generally designated 10. In one exemplary embodiment, the protective needle clip 10 (herein “needle clip” or “safety clip”) may be a unitary construction needle clip comprising three needle engaging arms 12, 14, 16 with each arm comprising a generally circular opening 20a, 20b, 20c. Viewing from the right side to the left side of the needle clip 10, the engaging arms may be referred to as the first engaging arm 12, the second engaging arm 16, which flows directly from the first engaging arm 12, and the third engaging arm 14, which flows directly from the second engaging arm, i.e., 12, 16, 14. However, for purposes of the following disclosure, the sequence 12, 14, 16 is used to designate the first arm 12, the second arm 14, and the third arm with the latter sequence, 12, 16, 14, being considered equivalent. The first needle engaging arm 12 is a distal most arm comprising a finger 18 extending at an angle therefrom and a first opening 20a located thereon. The first engaging arm 12 is configured to shield a needle tip to prevent accidental contact therewith, as further discussed below.

The third engaging arm 16 extends immediately from the first engaging arm 12 and an anchor mechanism 22 is disposed thereinbetween. In one exemplary embodiment, the anchor mechanism 22 comprises one or more tabs 24 protruding from a generally planar connecting base 26. The tabs 24 may be formed separately by bonding, riveting, or welding a material onto the connecting base 26. More preferably, the one or more tabs 24 may be made by cutting, notching, or stamping one or more sections of the connecting base 26 and rolling the one or more notched sections to create protruding elements from the generally planar surface of the connecting base. In one exemplary embodiment, the anchor mechanism 22 is positioned intermediate the first arm 12 and the third arm 16 and defining a distal base portion 28 and a proximal base portion 30 of the connecting base 26. In one exemplary embodiment, the distal base portion 28 may extend at a slight angle or an incline from the remaining portion of the connecting base 26 (FIG. 2).

The second engaging arm 14 extends from the third engaging arm 16 via a connecting top 32. The second engaging arm 14 is positioned in between the first engaging arm 12 and the third engaging arm 14.

In one exemplary embodiment, the needle clip 10 may be made by stamping an outline of the needle clip and the openings 20a, 20b, 20c from a thin planar sheet, such as a stainless steel sheet, and then folding the stamped material along the first fold 34, second fold 36, third fold 38, fourth fold 40, fifth fold 42, and sixth fold 44. The sequence of fold is not crucial to the embodiment disclosed and can vary depending on the manufacturing requirement and equipment involved in manufacturing the needle clip 10. The size or dimension of the needle clip 10 can also vary depending on its end use. For example, the needle clip 10 may be used in a catheter assembly by positioning the clip inside a catheter hub, on a standard hypodermic needle having a straight needle shaft, on a hypodermic needle having a spring launch mechanism, on a Huber needle, on a safety spinal needle, or on any needle where a needle tip is desired to be protected.

Referring now to FIG. 2, a semi-schematic side view of the safety clip 10 of FIG. 1 is shown in its pre-needle mount configuration, which is similar to an activated position (FIG. 4). In the pre-needle mount configuration, the connecting top portion 32 defines a first planar surface 46. The second needle engaging arm 14 and the third needle engaging arm 16 each extends a non-perpendicular angle from the first planar surface 46. In one exemplary embodiment, the second needle engaging arm 14 extends an angle −θ₁ (negative) from a second plane 48 and the third needle engaging arm 16 extends an angle θ₂ (positive) from a third plane 50, wherein the second plane and the third plane are both perpendicular to the first planar surface 46. In an alternative exemplary embodiment, the second engaging arm 14 can have an angle θ₁ (positive) and the third engaging arm 16 can have an angle −θ₂ (negative). In other words, the second arm 14 can have an angle ±θ₁ and the third arm 16 can have an angle ±θ₂, wherein any combination of angles are acceptable provided the overall configuration is adapted to both shield the needle tip and firmly secure the needle clip 10 onto the needle, as further discussed below. The connecting base 26 is positioned at an angle θ₃ from a fourth plane 52 and the first engaging arm 12 is positioned at an angle θ₄ from a fifth plane 54, which is generally parallel to the second 48 and third 50 planes.

In an activated position of one exemplary embodiment, an imaginary line 56 intersecting the second opening 20b of the second arm 14 and an imaginary line 58 intersecting the third opening 20c of the third arm 14, 16 intersect one another, as opposed to being generally coincident to one another when the needle clip is in a ready position, as further discussed below. Similarly, an imaginary line 60 intersecting the first opening of the first arm 12 intersect the other imaginary lines 56, 58 rather than being coincident with the other imaginary lines. Because the engaging arms 12, 14, 16 are positioned at an angle when in the pre-needle mount configuration and in the activated position (FIG. 4), the openings 20a, 20b, 20c each comprises an oval projection when viewed from a plane perpendicular to the vertical planes 48, 50, 54. Hence, while the openings 20a, 20b, 20c each has a generally circular projection when the angles θ₁, θ₂, θ₄ are approximately zero degree, they have a generally oval projection when the angles θ₁, θ₂, θ₄ are a few degrees from zero, such as greater than about ±3 degrees.

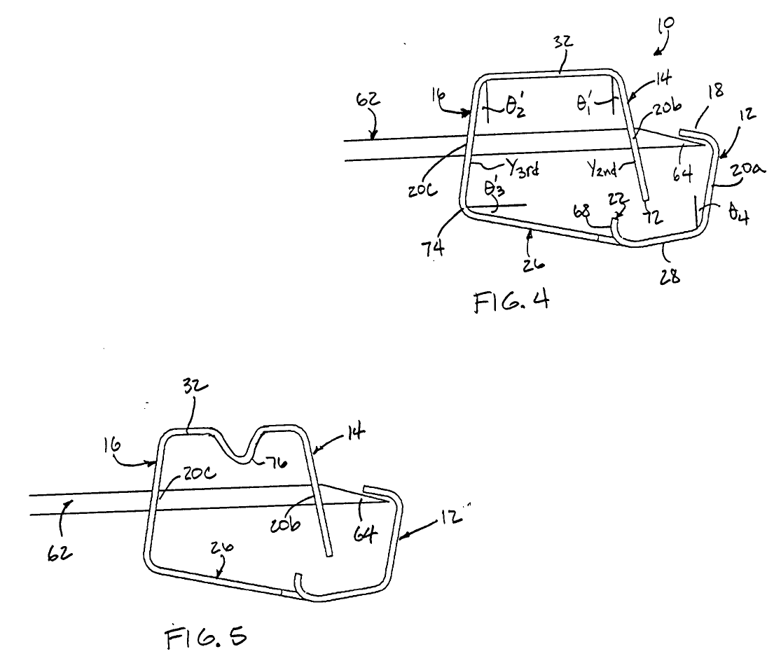

Referring now to FIG. 3, a semi-schematic side view of the needle clip 10 of FIG. 2 is shown in a loaded or ready position over a hypodermic needle 62 comprising a needle tip 64, which may include a non-coring needle tip (i.e., such as a Huber needle tip) or a coring needle tip. In the loaded position, the second engaging arm 14 is placed into abutting contact with the anchor mechanism 22. In one exemplary embodiment, this may be accomplished by applying a force on the second engaging arm 14 and a force on the connecting base 26 to compress the same until the tip 66 of the second engaging arm 14 comes over the anchor mechanism 22 and into abutting relationship with the active surface 68 of the anchor mechanism. The needle clip 10 may then be held in the ready position by inserting the needle 62 through the openings 20a, 20b, 20c, which are generally coincident to one another, to keep the first needle engaging arm 12 in a biased position to then retain the remaining arms 14, 16 and the needle clip in a biased position.

In one exemplary embodiment, when the needle clip 10 is in the ready position, the various angles θ₁, θ₂, θ₂, θ4 are at approximately zero degree. However, as readily apparent by a person of ordinary skill in the art, the angles can vary by a few degrees by altering the relative dimensions between the openings 20a, 20b, 20c of the engaging arms 12, 14, 16 and the outside diameter of the needle 62. For example, if the openings 20a, 20b, 20c are each larger than the outside diameter of the needle 62, then the larger relative dimensions of the openings allow the openings to project slightly ovally while over the needle without being physically obstructed by the needle. Thus, this slightly oval projection will allow the angles θ₁, θ₂, θ₂, θ4 to be plus/minus a few degrees when in the ready position.

Indeed, in one exemplary embodiment, the openings 20a, 20b, 20c have a first generally oval configuration when in the ready position over the needle and a larger oval configuration when in the activated position (FIG. 4) over the needle. Under this configuration, the angles θ₁, θ₂, θ₂, θ4 all have an integer value when in the ready position over the needle. Alternatively, the openings 20a, 20b, 20c have a generally circular configuration while over the needle and have an oval configuration when in the activated position. Under this configuration, the angles θ₁, θ₂, θ₂, θ4 are about zero degree when in the ready position and the engaging arms 12, 14, 16 are generally straight. As readily apparent by a person of ordinary skill in the art, under any configuration, the openings 20a, 20b, 20c should be sufficiently larger than the outside diameter of the needle 62 to enable the needle tip 64 to move from a distal position in which the needle tip extends beyond the first engaging arm 12 to a proximal position in which the needle tip moves proximal of the first engaging arm 12. However, the openings 20a, 20b, 20c should also be limited in size so as to provide a friction grip against the exterior surface of the needle 62 when the needle clip is in the activated position.

Referring now to FIG. 4, a semi-schematic side view of the needle clip in an activated position for shielding the needle tip 64 is shown. The needle clip 10 transitions from the ready position (FIG. 3) to the activated position when the needle 62 moves from a distal position in which the needle tip 64 extends beyond the first engaging arm 12 (FIG. 3) to a proximal position in which the needle tip 64 moves proximal of the first engaging arm 12. As the needle 62 moves between these two positions, the biased on the needle clip 10 is released and at least a portion of the first engaging arm 12 moves radially outwardly away from the needle axis. In one exemplary embodiment, the radial outward movement of the first engaging arm 12 is delimited by the finger 18 moving radially towards the needle tip 64 and abutting the needle tip. In an alternative embodiment, the radial movement of the first engaging arm 12 is delimited by the original angular position of the connecting base 26, which is described above as being related to angle θ₃. Still alternatively, the first engaging arm 12 may have a length or a height such that in the activated position, the finger 18 remains spaced apart from the needle tip 64 and the gap 70 between the finger 18 and the needle tip is greater than as shown. If the finger 18 abuts a portion of the needle tip 64, the angle defined by the angular position between the third engaging arm 16 and the connection base 26 may be θ′₃, which may be different than θ₃ (FIG. 2).

During the same needle tip travel from a distal position to a proximal position just proximal of the first engaging arm 12, the needle clip 10 axially expands. In one exemplary embodiment, the second engaging arm 14 axially expands at the second arm tip 66 by approximately a distance of Y_{2nd}*Tan θ′₁, where Y_{2nd} is the length of the second arm 14 measured from a point at approximately the intersection with the connecting top 32 to a point approximately at the tip 66 of the second arm 14. Similarly, the third engaging arm 16 axially expands at the third arm tip 74 by approximately a distance of Y_{3rd}*Tan θ′₂, where Y_{3rd} is the length of the third arm 16 measured from a point approximately at the intersection with the connecting top 32 to a point at approximately the intersection with the connecting base 26. As further discussed below, angles θ′₁ and θ′₂ may be different than θ₁ and θ₂, respectively, due to the engaging arms 14, 16 remaining in a biased state. This allows the spring clip to maintain a grip on the needle 62 when in the activated position. As readily apparent, the axial expansions are described in absolute terms without reference to the angle orientation.

Still referring to FIG. 4, when the needle clip 10 expands and the second and third engaging arms 14, 16 move as described above, the openings 20b, 20c on the second and third engaging arms 14, 16 project form a generally circular cross-section to a generally oval cross section. Alternatively, the openings 20b, 20c may project from a smaller oval cross section to a relatively larger oval cross-section depending on the starting angles. During the transition between the ready position and the activated position, an upper and lower portions of each of the openings 20b, 20c contact the exterior surface of the needle 62 and wedge against the exterior surface by a force corresponding to the bias or load defined by the differences between [θ′₁:θ₁]; [θ′₂:θ₂]; and the resiliency of the needle clip 10, which may be influenced by the particular material selected and the thickness utilized. In one exemplary embodiment, the amount of force exerted by the openings 20b, 20c on the exterior surface of the needle is such that the spring clip 10 remains firmly secured on the needle and further proximal travel by the needle tip 64 relative to the first engaging arm 12 is restricted. Preferably, this restriction is sufficient to withstand a normal retraction force on the needle 62 to withdraw the needle relative to the needle clip 10. In an exemplary embodiment, this restriction may be increased by providing a relatively stronger spring clip, such as by using different material, by selecting a thicker material, or by incorporating additional biasing means, as further discussed below. A purposeful retraction force on the needle relative to the needle clip 10, however, may still overcome the friction between the openings 20b, 20c and the needle exterior surface and causes the needle clip 10 to separate from the needle 62.

Referring now to FIG. 5, an alternative needle clip 10′ comprising a coiled portion 76 formed on the connecting top 32 is shown. When incorporated, the coiled portion 76 may increase the bias force on the second and third engaging arms 14, 16 to increase the wedging force between the upper and lower portions of the openings 20b, 20c and the exterior surface of the needle 62. Alternatively or in addition to the coiled portion 76, a notch or a slit in the needle near the needle tip 64 may be incorporated to interact with an edge of the opening 20b of the second engaging arm 14. When incorporated, the notch or slit in the needle 62 can further restrict or prevent the needle clip 10 from separating from the needle.

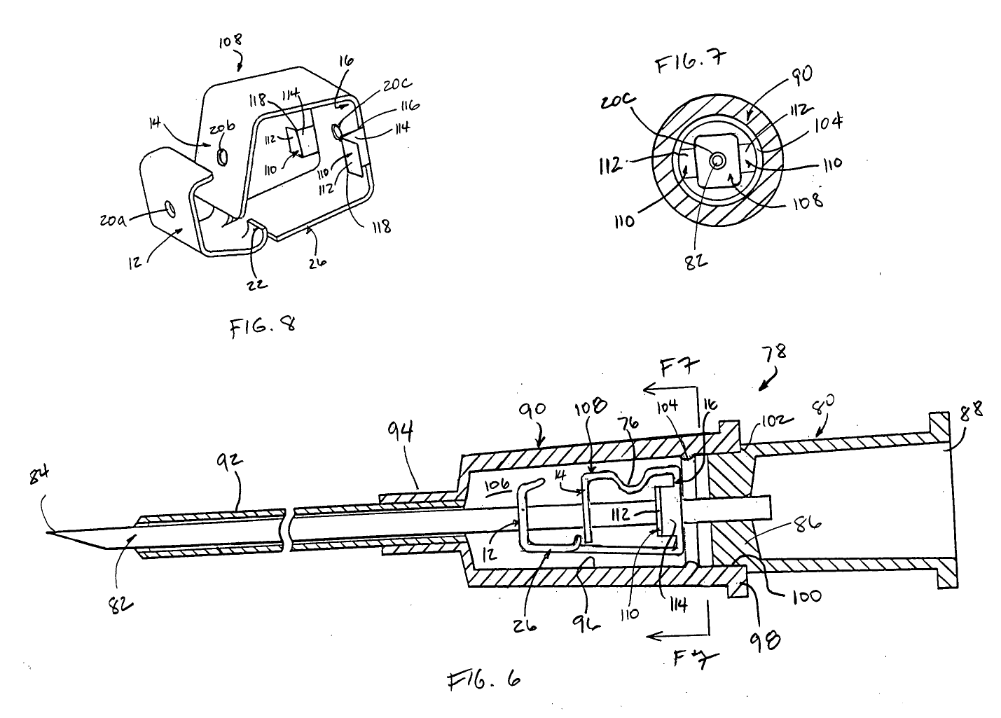

FIG. 6 is a semi-schematic cross-sectional side view of a catheter needle assembly 78 provided in accordance with aspects of the present invention. In one exemplary embodiment, the catheter needle assembly 78 comprises a needle hub 80 comprising a needle 82 having a needle tip 84 extending from a distal end 86 of the needle hub. A proximal end opening 88 is provided for injecting and/or collecting fluids via the needle lumen.

A catheter hub 90 comprising a flexible catheter tube 92 extending from a distal end 94 of the catheter hub is shown mechanically coupled to the distal end 86 of the needle hub 80. In one exemplary embodiment, the interior surface 96 of the proximal end 98 of the catheter hub 90 is frictionally engaged to the exterior surface 100 of the distal end 86 of the needle hub. A shoulder or a notch 102 may be incorporated on the exterior surface 100 of the needle hub to delimit the extent to which the catheter hub 90 extends over the exterior surface of the needle hub 80.

A bump 104 on the interior surface 96 extending into the interior cavity 106 of the catheter hub 90 may be provided for manipulating the spring clip 108 positioned within the interior cavity 106. The bump 104 may comprise two or more bumps formed along the interior surface of the catheter hub or a continuous ring. The bump 104 manipulates the spring clip 108 by interacting with one or more retaining wings 110 on the spring clip 108 to control movement of the spring clip. As readily apparent to a person of ordinary skill in the art, when the needle hub 80 separates from the catheter hub 90 following an insertion, as further discussed below, the bump 104 abuts the one or more retaining wings 110 to retain the spring clip 108 with the catheter hub 90 and allows the spring clip to move relative to the needle 82 and the needle hub 80. The spring clip 108 is retained by the bump 104 and remains within the interior cavity 106 of the catheter hub 90 until the needle tip 84 moves just proximal of the first engagement arm 12 of the spring clip, whereupon the spring clip activates, attaches to the needle 82 to shield the needle tip 84, and separates from the catheter hub 90. In one embodiment, the bump 104 is positioned further distally within the interior cavity 106 of the catheter hub 90 such that the bump 104 contacts the one or more retaining wings 110 when in the ready configuration shown.

In one exemplary embodiment, the spring clip 108 for use in conjunction with the catheter needle assembly 78 is substantially the same as the spring clip shown with reference to FIGS. 1-5 with the exception of the one or more retaining wings 110. Thus, like the earlier described spring clip, the spring clip 108 shown includes a first needle engaging arm 12, a second needle engaging arm 14, and a third needle engaging arm 16 with each engaging arm comprising an opening 20a, 20b, 20c (not shown). Optionally, a coiled spring section 76 may be incorporated to increase the wedging force on the needle, as discussed above with reference to FIG. 5.

The catheter hub assembly 78 may be assembled by first sliding the spring clip 108 over the needle 82. The catheter tube 92 of the catheter hub 90 is then slid over the needle 82. Just prior to mechanically joining the interior surface 96 of the catheter hub with the exterior surface 100 of the needle hub 80, the spring clip 108 is pushed distally past the bump 104 with a pushing rod or a stick or similar objects. Alternatively, an extension may be formed on the distal end 86 of the needle hub 80 so that the extension automatically pushes the spring clip 108 past the bump 104 when the catheter hub 90 is mated with the needle hub 80.

Referring now to FIG. 7, an end view of the catheter needle assembly 78 of FIG. 6 is shown taken along line F7-F7. As shown, the retaining wings 110 of the needle clip 108 comprise a first retaining section 112 and a second retaining section 114 (FIG. 6). The first retaining section 112 is configured to abut the bump 104 on the interior cavity of the catheter hub. The abutment is such that a surface of the first retaining section 112 of the one or more retaining wings 110 contact a side of the bump 104. The contact allows the spring clip 108 to be retained by the catheter hub 90 and allows the spring clip to move relative to the needle hub 80 when the catheter hub and the needle hub are separated from one another, as further discussed below.

Referring now to FIG. 8, a semi-schematic perspective view of the spring clip 108 of FIGS. 6 and 7 is shown without the coiled portion 76. Referring specifically to the one or more retaining wings 110, in one exemplary embodiment, the one or more retaining wings 110 comprise a first resilient angle 116 formed between the second retaining section 114 and the third engaging arm 16, and a second resilient angle 118 formed between the second retaining section 114 and the first retaining section 112. The first and second resilient angles 116, 118 may comprise an acute angle, an obtuse angle, or a combination thereof. The resilient angles 116, 118 allow the one or more retaining wings 110 to flex about the axes formed by the two angles. The dimension and the thickness of the one or more retaining wings 110 should be such that the one or more retaining wings 110 will flex, bend, or give when a separation force is exerted by the bump 104 via a pulling force on the needle hub to separate the needle hub from the catheter hub, as further discussed below.

Referring again to FIGS. 6 and 7 in addition to FIG. 8, the spring clip 108 may be activated to block the needle tip 84 of the needle 82 following an injection by first retracting the needle hub 80 relative to the catheter hub 90. This is performed by moving the needle hub proximally (to the right of FIG. 6). As the needle hub 80 moves proximally relative to the catheter hub 90, the bump 104 interacts with the one or more retaining wings 110 to retain the spring clip 108 within the interior cavity 106 of the catheter hub. As the needle hub continues 80 to move proximally until the needle tip 84 moves proximal of the first engaging arm 12, the connecting base 26 of the spring clip is released and moves radially outwardly relative to the needle 82. This in turn releases the second engaging arm 14 from the anchor mechanism 22 and allows the second engaging arm 14 and the third engaging arm 16 to axially expand. As previously discussed, the axial expansion of the second engaging arm 14 and the third engaging arm causes the openings 20b and 20c located on the second and third engaging arms 14, 16 to wedge against the exterior surface of the needle to secure the spring clip to the needle.

When the openings 20b, 20c of the second and third engaging arms 14, 16 are wedged against the exterior surface of the needle, the spring clip 108, via the wedging force, is retained on the needle 82 as shown in FIG. 4. At this point, further proximal movement of the needle hub 80 imparts a separation force on the one or more retaining wings 110 via the bump 104 pushing against the first retaining section 112 of the one or more retaining wings 110. This separation force causes bending or flexing of the one or more retaining wings 110. Because the wedging force exerted by the openings 20b, 20c on the exterior surface of the needle is greater than the force to flex the one or more retaining wings 110, the proximal movement of the needle hub 80 flexes the one or more retaining wings 110 sufficiently apart to permit the bump to move proximally past the one or more retaining wings 110 to allow the catheter hub to separate from the spring clip.

Although the preferred embodiments of the invention have been described with some specificity, the description and drawings set forth herein are not intended to be delimiting, and persons of ordinary skill in the art will understand that various modifications may be made to the embodiments discussed without departing from the scope of the invention, and all such changes and modifications are intended to be encompassed within the appended claims. Various changes to the needle clip comprising a radially expanding portion and at least one axially expanding portion when the transformed from a ready position to an activated position may be made without deviating from the spirit and scope of the present invention. For example, the dimensions of the spring clip can vary depending on the particular hypodermic needle assembly used in combination with the spring clip, the material selection can vary, the angles can vary, and the spring clip can be made by assembling or bonding different components together instead of from a unitary construction. Other changes include incorporating a different anchor mechanism, such as a bump added by attaching a rivet or a depositing material onto the connecting base, provide curvatures for the first arm, second arm, connecting base, etc., and changing the bias configuration from radial expansion to radial compression or a combination thereof for the second and third arms when activated. Still other changes may include using a resilient spring to advance the needle clip towards the needle tip to shield the needle tip, wedging the spring clip inside a catheter hub to be moved by the catheter hub towards the needle tip, and wedging the spring clip inside a collar of a Huber needle to be moved by the collar towards the needle tip of the Huber needle. Accordingly, many alterations and modifications may be made by those having ordinary skill in the art without deviating from the spirit and scope of the invention.

1. A protective needle clip comprising a first engaging arm, a connecting base extending at a first angle from the first engaging arm, a second engaging arm extending at a second angle from the connecting base, a connecting top extending at a third angle from the second engaging arm, and a third engaging arm extending at a fourth angle from the connecting top; wherein each of the first, second, and third engaging arms comprises an opening, and wherein at least a portion of the third engaging arm is disposed in between the first engaging arm and the second engaging arm.

2. The protective needle clip of claim 1, further comprising an anchor mechanism disposed in between the first engaging arm and the second engaging arm for abutting with the third engaging arm.

3. The protective needle clip of claim 1, further comprising a finger portion extending at a fifth angle from the first engaging arm.

4. The protective needle clip of claim 1, wherein the first, second, third, and fourth angles are approximately the same within plus or minus three degrees from one another when a needle passes through the openings of the first, second, and third engaging arms.

5. The protective needle clip of claim 1, wherein the connecting base comprises a tapered portion defining a dimension smaller than a non-tapered portion of the connecting base.

6. The protective needle clip of claim 2, wherein the anchor portion comprises a bend formed from a cut out on the connecting base.

7. The protective needle clip of claim 1, wherein the connecting top comprises a coiled portion formed by bending a portion of the connecting top.

8. The protective needle clip of claim 1, wherein the second and third engaging arms each comprises a portion that moves axially relative to a needle having a needle axis and the first engaging arm comprises a portion that moves radially relative the needle axis.

9. The protective needle clip of claim 1, wherein the openings each has an inside diameter that are the same.

10. A protective needle clip comprising an engaging arm comprising an opening extending from a connecting base, the engaging arm and the connecting base comprising a ready position, which is a position in which a needle comprising a needle tip passes through the opening of the engaging arm and biasing the connecting base and the at least a portion the engaging arm radially inwardly relative to the needle, and an activated position, which is the position in which the needle tip moves proximal of the opening and the connecting base and the at least a portion of the engaging arm move radially outwardly away from the needle.

11. The protective needle clip of claim 10, further comprising a second engaging arm and a third engaging arm, the second and third engaging arms each comprises an opening and wherein the needle passes through the openings of the second and third engaging arms when the needle clip is in the ready position.

12. The protective needle clip of claim 10, further comprising a second engaging arm and a third engaging arm, the second and third engaging arms each comprises an opening and wherein the needle passes through the openings of the second and third engaging arms when the needle clip is in the retracted position.

13. The protective needle clip of claim 10, further comprising a second engaging arm extending from the connecting base, a connecting top extending from the second engaging arm, and a third engaging arm extending from the connecting top.

14. The protective needle clip of claim 13, wherein the second and the third engaging arms each comprises an opening and wherein the needle extends through all three openings when the protective needle clip is in the ready position.

15. The protective needle clip of claim 14, wherein the needle extends through the opening of the second and third engaging arms when the protective needle clip is in the retracted position.

16. The protective needle clip of claim 15, wherein the protective needle clip is firmly secured to the needle by at least a portion of each of the openings of the second and third engaging arms wedging against an exterior surface of the needle.

17. The protective needle clip of claim 15, wherein the openings of the second and third engaging arms have an oval projection when the needle clip is in the retracted position.

18. A method for shielding a needle tip of a hypodermic needle comprising:
moving the hypodermic needle relative to a protective needle clip such that the protective needle clip moves from a proximal position on the needle to a distal position on the needle, the protective needle clip comprising a first engaging arm extending from a connecting base, a second engaging arm extending from said connecting base, a connecting top extending from the second engaging arm, and a third engaging arm extending from the connecting top; wherein the first, second, and third engaging arms each comprises an opening and wherein the hypodermic needle extends through the three openings;
moving the needle tip of the hypodermic needle proximal of the opening of the first engaging arm; and
allowing at least a portion of the first engaging arm to move radially outwardly away from the needle.

19. The method of claim 18, wherein the second and third engaging arms are in an axially compressed configuration when the needle extends through the three openings.

20. The method of claim 19, wherein the second and third engaging arms axially expand when the needle tip of the hypodermic needle moves proximal of the opening of the first engaging arm.

21. The method of claim 18, wherein the needle clip comprises a ready position, and wherein at least two of the three engaging arms are parallel to one another when the needle clip is in the ready position.

22. The method of claim 18, wherein the protective needle clip further comprises an anchor mechanism positioned on the connecting base.

23. The method of claim 22, wherein the anchor mechanism comprises a cut-out and a bend in the cut-out.

24. The method of claim 18, wherein the connecting top comprises a coiled portion.

25. A catheter needle assembly comprising:
catheter hub comprising an interior surface defining an interior cavity, an open proximal end, and a distal end having a catheter tube extending therefrom, the catheter tube comprising a lumen;
a needle hub comprising an open proximal end and a distal end having a needle comprising a needle tip extending therefrom;
a ready position in which the needle extends through the lumen of the catheter tube, the needle tip extends beyond a distal end of the catheter tube; and a portion of the catheter hub's interior surface in mechanical engagement with an exterior surface of the needle hub; and
a spring clip positioned inside the interior cavity of the catheter hub comprising a first engagement arm, a second engagement arm, and a third engagement arm, each arm having an opening and the needle extending through each of the three openings;
and a raised section on the spring clip for contacting the second engagement arm and biasing the second engagement arm.

26. The catheter needle assembly of claim 25, wherein a bump in the interior cavity of the catheter hub is in contact with a portion of the spring clip.

27. The catheter needle assembly of claim 25, further comprising a ring on an interior cavity of the catheter hub.

28. The catheter needle assembly of claim 25, wherein the spring clip further comprises a flexible retaining wing adapted to contact a portion of the interior cavity of the catheter hub.

29. The catheter needle assembly of claim 25, wherein the first engagement arm moves radially outwardly relative to the needle when the needle tip moves proximal of the opening on the first engagement arm.

30. The catheter needle assembly of claim 25, wherein the spring clip further comprises a coiled section.
